(19) United States
(12) Patent Application Publication
(10) Pub. No.: US 2005/0164227 A1
(43) Pub. Date: Jul. 28, 2005

(54) Method for preparing semiconductor nanocrystals having core-shell structure

(75) Inventors: Atsuhiko Ogura, Tsuchiura-shi (JP); Eui-chul Kang, Tsukuba-shi (JP)

Correspondence Address: DARBY & DARBY P.C., P. O. BOX 5257, NEW YORK, NY, 10150-5257, US

(73) Assignee: NOF Corporation, Tokyo (JP)

(21) Appl. No.: 10/913,305

(22) Filed: Aug. 5, 2004

(30) Foreign Application Priority Data
Aug. 8, 2003 (JP) P2003-289484

(51) Int. Cl. G01N 33/53

(52) U.S. Cl. 435/6; 435/7.1

(57) ABSTRACT

The invention relates to a method for producing semiconductor nanocrystals with a core-shell structure and the semiconductor nanocrystals obtained by the method, which enables continuous production in a compact system. The method includes (1) passing a stock solution of a core component such as CdSe through a first hollow microchannel having an inner diameter of 1 to 1000 μm at a predetermined constant flowrate to form cores at 250 to 350° C., (2) passing a stock solution of a shell component such as ZnS through a second microchannel, and (3) passing the core stream merged with the shell component stream through a third microchannel at a predetermined constant flow rate to epitaxially grow the shell component on the cores at 100 to 250° C. to thereby form a core-shell structure. The microchannels communicate with each other, and step (3) is performed consecutively with steps (1) and (2).

### FIELD OF ART

The present invention relates to a method for producing semiconductor nanocrystals of a nanometer size, in particular to a method for continuously producing semiconductor nanocrystals with a core-shell structure, using cylindrical microchannels.

### BACKGROUND ART

Semiconductor nanocrystals are known to have optical characteristics that are different from those of bulk semiconductors. For example, (1) the nanocrystals are capable of coloring and emitting light of various wavelengths depending on their size, (2) the nanocrystals have a broad absorption range, and excitation light of a single wavelength can excite various sizes of crystals to emit light, (3) the fluorescence spectrum of the nanocrystals is highly symmetric, and (4) the nanocrystals have superior durability and anti-fading property, compared to organic dyes. The semiconductor nanocrystals have recently been studied intensively for applications not only in optics and electronics such as display elements and recording materials, but also in fluorescent markers and biological diagnosis.

It is reported in U.S. Pat. No. 6,207,229 that semiconductor nanocrystals are produced by a batch method in a glass container. This method, however, provides particularly poor reproducibility of semiconductor nanocrystals emitting short-wavelength fluorescence, and may be hard to scale up due to its thermal history.

It is proposed in JP-2003-25299-A that semiconductor nanocrystals of a uniform particle size are produced by means of optical etching. However, this method requires irradiation equipment and complicated procedures.

On the other hand, Size-Controlled Growth of CdSe Nanocrystals in Microfluidic Reactors, Nano Lett., 3(2); p199 (2003) reports CdSe nanocrystals produced by means of cylindrical microchannels, and JP-2002-79075-A reports CdS nanocrystals. In the former article, it is reported that CdSe nanocrystals of relatively high quality are produced by passing a Cd/Se stock solution through heated microchannels formed in a pattern on a glass substrate. In the latter publication, it is reported that CdS nanocrystals are produced by preparing reverse micelle solutions of cadmium nitrate and sodium sulfide, respectively, and reacting these solutions by contact catalysis in a tubular flow reactor.

The methods employing microchannels, wherein continuous reaction is possible, are expected to provide potentially high productivity, to enable instant control of a reaction temperature, and to produce nanocrystals of a desired particle size or fluorescence wavelength with excellent reproducibility.

However, both of the above reports relate to methods for producing semiconductor nanocrystals of a single component, and no report has been made on a method for continuously producing, through microchannels, semiconductor nanocrystals with a core-shell structure, wherein semiconductor is coated with semiconductor to form a composite.

Conventional semiconductor nanocrystals of a single component often have problems of decreased fluorescence intensity or even quenching caused by oxidation or optical etching of the nanocrystal surface, or isolation of ligand. It is thus necessary to improve the fluorescence intensity of semiconductor nanocrystals and to stabilize their light emission behavior irrespective of external environmental changes, by giving semiconductor nanocrystals a core-shell structure by coating a core semiconductor with another semiconductor with a larger band gap.

In this regard, Margaret A., et al., J. Phys. Chem., 100, p468 (1996) reports a method for discontinuously producing ZnS-capped CdSe having a core-shell structure, wherein CdSe cores are prepared by a batch reaction, and a zinc/sulfur stock solution is added thereto.

Thus there are demands for a method for continuously producing semiconductor nanocrystals having a core-shell structure.

### SUMMARY OF THE INVENTION

It is an object of the present invention to provide a method for producing semiconductor nanocrystals with a core-shell structure that enables continuous production of the nanocrystals.

It is another object of the present invention to provide a method for producing semiconductor nanocrystals with a core-shell structure that enables continuous production of the nanocrystals and requires only a compact production system.

It is yet another object of the present invention to provide semiconductor nanocrystals having a particle size of 1 to 10 nm and a full width at half maximum of the fluorescence spectrum of not wider than 30 nm.

According to the present invention, there is provided a method for producing semiconductor nanocrystals with a core-shell structure comprising the steps of:
(1) passing a stock solution of a core component consisting of CdX, wherein X stands for S, Se, or Te, through a first hollow microchannel having an inner diameter of 1 to 1000 μm at a constant flow rate of 0.25 to 25 ml/min to form cores of the semiconductor nanocrystals in a temperature range of 250 to 350° C.,
(2) passing a stock solution of a shell component consisting of ZnR, wherein R stands for S, Se, Te, or O, through a second hollow microchannel having an inner diameter of 1 to 1000 μm, and
(3) passing a stream of said cores formed through the first microchannel merged with a stream of said shell component from the second microchannel, through a third hollow microchannel having an inner diameter of 1 to 1000 μm at a constant flow rate of 0.5 to 50 ml/min to epitaxially grow said shell component on said cores in a temperature range of 100 to 250° C., to thereby form a core-shell structure,
wherein said first, second, and third microchannels communicate with each other, and
wherein said step (3) is performed consecutively to said steps (1) and (2).

According to the present invention, there is also provided semiconductor nanocrystals obtained by the above method, said nanocrystals having a core consisting of CdX, wherein X stands for S, Se, or Te, and a shell consisting of ZnR, wherein R stands for S, Se, Te, or O, said nanocrystals having a particle size of 1 to 10 nm, and a full width at half maximum of the fluorescence spectrum of not wider than 30 nm.

### BRIEF DESCRIPTION OF THE DRAWINGS

FIG. 1 is a schematic view of a system for producing semiconductor nanocrystals with a core-shell structure in a cylindrical reaction field.

FIG. 2 is a graph showing the fluorescence spectra of semiconductor nanocrystal samples prepared in Examples 1 to 5.

FIG. 3 is a graph showing the full widths at half maximum (FWHM) and peaks of the fluorescence spectra shown in FIG. 2.

### PREFERRED EMBODIMENTS OF THE INVENTION

The present invention will now be explained in detail.

The present invention is a method for continuously producing semiconductor nanocrystals having a core of CdX, wherein X stands for S, Se, or Te, namely a core of CdS, CdSe, or CdTe, and a shell of ZnR, wherein R stands for S, Se, Te, or O, namely a shell of ZnS, ZnSe, ZnTe, or ZnO. For example, when the core is made of CdS or CdSe, and the shell is made of ZnS, semiconductor nanocrystals that emit light in the visible light range are obtained.

In the method of the present invention, step (1) is performed, wherein a stock solution of a core component composed of CdX is passed through the first hollow microchannel having an inner diameter of 1 to 1000 μm at a constant flow rate of 0.25 to 25 ml/min to form cores of the semiconductor nanocrystals in a temperature range of 250 to 350° C.

If the inner diameter of the first microchannel, as well as the second and third microchannels to be discussed later, is smaller than 1 μm, the fluid delivery pump is excessively burdened, whereas if larger than 1000 μm, influence of the diffusing factor is large, which broadens the particle size distribution of the resulting semiconductor nanocrystals.

The microchannels used in the present invention may be made of any materials, as long as the material is chemically inert, and will not fuse or degenerate in the temperature range of 100 to 350° C., for fulfilling its purpose to provide a reaction field. For example, metals such as stainless steel or aluminum; or inorganic materials such as silica may preferably be used. The microchannels may preferably be arranged linearly, but may also be arranged in a spiral shape for making the production system compact.

The length of the first microchannel, as well as the third microchannel to be discussed later, may preferably be 0.1 to 10 m. With a length exceeding 10 m, the fluid delivery pump is excessively burdened, whereas with a length of shorter than 0.1 m, reproducible results are hard to be achieved.

The stock solution of a core component used in step (1) contains a semiconductor material selected from the group consisting of organic cadmium, salts of an organic acid and cadmium, selenium, tellurium, bis(trimethylsilyl)sulfide, and mixtures thereof. For example, for CdSe cores, the semiconductor material is selected and blended so that cadmium and selenium are present at an equal molar ratio.

The organic cadmium and the salts of an organic acid and cadmium are not particularly limited, and dimethyl cadmium and cadmium stearate may preferably be used.

The semiconductor material may be a commercially available product. However, since the purity of the material has an impact on the fluorescence characteristics of the resulting semiconductor nanocrystals, it is preferred to use a product of as high purity as available, usually not lower than 99% purity.

The stock solution of a core component contains a reaction solvent for dissolving the semiconductor material. Such a solvent may be at least one solvent selected from the group consisting of alkylphosphines such as trioctylphosphine and tributylphosphine; alkylphosphine oxides such as trioctylphosphine oxide and tributylphosphine oxide; alkyl amines such as dioctyl amine and hexadecyl amine; and mixtures thereof. Of these examples, combinations of alkylphosphine oxides and alkyl amines are particularly preferred.

In preparing the stock solution of a core component, the semiconductor material is dissolved in the reaction solvent so that the cadmium content in the stock solution is usually 1 μmol/ml to 1 mmol/ml, preferably 5 μmol/ml to 100 μmol/ml, most preferably 10 μmol/ml to 50 μmol/ml, in terms of the cadmium content in the semiconductor material. At a cadmium content of lower than 1 μmol/ml, a large amount of solvent is disadvantageously required for preparation of the cores, whereas at a cadmium content of higher than 1 mmol/ml, high quality semiconductor nanocrystals are hard to be obtained.

In step (1), if the flow rate of the stock solution of a core component is slower than 0.25 ml/min or faster than 25 ml/min, semiconductor crystals having a particle size of 1 to 10 nm and emitting light in the visible light range are hard to be obtained.

In step (1), if the temperature for forming the cores is lower than 250° C., the semiconductor nanocrystals cannot be matured sufficiently. If the temperature is higher than 350° C., the crystal grain size of the cores is hard to be controlled.

The particle size of the cores formed in step (1) is preferably 1 to 10 nm for efficient light emission of the resulting semiconductor nanocrystals in the visible light range.

In the method of the present invention, step (2) is performed, wherein a stock solution of a shell component composed of ZnR is passed through the second hollow microchannel having an inner diameter of 1 to 1000 μm.

The stock solution of a shell component used in step (2) contains a semiconductor material selected from the group consisting of organic zinc, salts of an organic acid and zinc, selenium, tellurium, bis(trimethylsilyl)sulfide, and mixtures thereof. For example, for a ZnS shell component, the semiconductor material is selected and blended so that zinc and sulfur are present at an equal molar ratio.

The organic zinc and the salts of an organic acid and zinc are not particularly limited, and diethyl zinc and zinc stearate may preferably be used.

The semiconductor material may be a commercially available product. However, since the purity of the material has an impact on the fluorescence characteristics of the resulting semiconductor nanocrystals, it is preferred to use a product of as high purity as available.

The stock solution of a shell component contains a reaction solvent for dissolving the semiconductor material. Such a solvent may be selected from those mentioned for the stock solution of a core component. Practically preferred is a solvent which is in a liquid form at room temperature, for example, at least one solvent selected from the group consisting of alkylphosphines such as trioctylphosphine and tributylphosphine.

In preparing the stock solution of a shell component, the semiconductor material is dissolved in the reaction solvent so that the zinc content in the stock solution is usually 1 μmol/ml to 1 mmol/ml, preferably 5 μmol/ml to 100 μmol/ml, most preferably 10 μmol/ml to 50 μmol/ml, in terms of the zinc content in the semiconductor material. At a zinc content of lower than 1 μmol/ml, a large amount of solvent is disadvantageously required for preparation of the semiconductor nanocrystals with a core-shell structure, whereas at a zinc content of higher than 1 mmol/ml, high quality semiconductor nanocrystals are hard to be obtained.

In step (2), a preferred flow rate of the stock solution of a shell component is usually 0.25 to 25 ml/min. At the flow rate of slower than 0.25 ml/min, the productivity is disadvantageously lowered, whereas at the flow rate of faster than 25 ml/min, the shell component is not allowed to grow sufficiently.

In the method of the present invention, step (3) is performed, wherein a stream of the cores formed through the first microchannel merged with a stream of the shell component from the second microchannel is passed through the third hollow microchannel having an inner diameter of 1 to 1000 μm at a constant flow rate of 0.5 to 50 ml/min to epitaxially grow the shell component on the cores in a temperature range of 100 to 250° C., thereby forming a core-shell structure.

In step (3), if the flow rate of the merged stream is slower than 0.5 ml/min, the productivity is lowered, whereas if faster than 50 ml/min, the shell component is not allowed to grow sufficiently. Further, if the temperature for epitaxially growing the shell component is lower than 100° C., the semiconductor forming the shell is not matured sufficiently, whereas if higher than 250° C., undesired by-products are generated.

In the method of the present invention, the first, second, and third microchannels for performing steps (1) to (3) communicate with each other, and step (3) is performed consecutively to steps (1) and (2). Thus, the semiconductor nanocrystals having a desired core-shell structure may be produced continuously.

The present invention will now be explained with reference to embodiments taken in conjunction with the attached drawings.

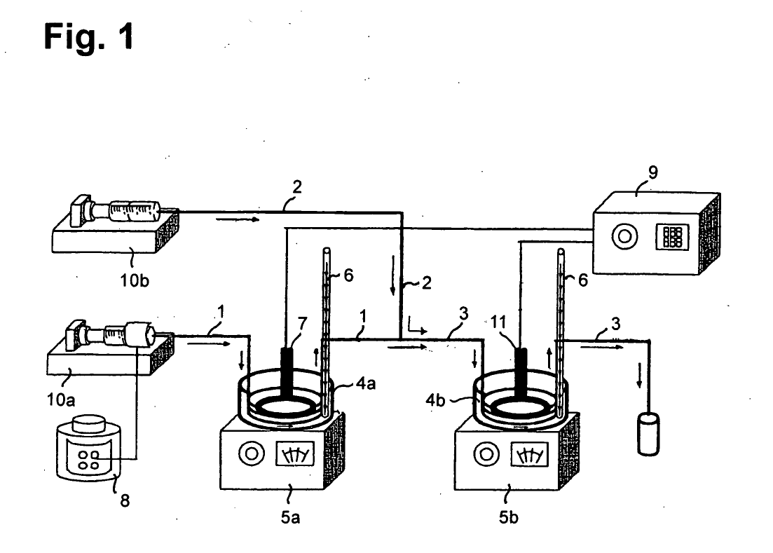

FIG. 1 illustrates an example of a system for producing the semiconductor nanocrystals according to the present invention, wherein numeral 1 refers to a first microchannel, 2 to a second microchannel, and 3 to a third microchannel. One end of the first microchannel 1 is connected to a pump 10a equipped with a transformer 8 for delivering the stock solution of a core component, and one end of the second microchannel 2 is connected to a pump 10b for delivering the stock solution of a shell component. The other ends of the first and second microchannels 1 and 2 are in communication with the third microchannel 3 so that the fluids in the first and second microchannels merge in the third microchannel 3. The other end of the third microchannel is a discharge port for the produced semiconductor nanocrystals. Here, the pumps 10a and 10b are selected from pumps that are capable of feeding each stock solution into the microchannel 1 or 2 at a constant flow rate, usually in a range of 0.1 to 10 ml/min, under precise control. Examples of such a pump may include a syringe pump and a liquid delivery pump for high performance liquid chromatography.

The first microchannel 1 is arranged to pass through an oil bath 4a disposed on a stirrer 5a for temperature control of a predetermined section of the microchannel 1. In the oil bath 4a, an immersion heater 7 for cores and a thermometer 6 connected to a temperature controller 9 are disposed.

Though not shown in the drawings, the first microchannel 1 is also equipped with a heating mechanism, such as a ribbon heater or a thermostatic water circulating device. This heating mechanism is used because trioctylphosphine oxide and hexadecyl amine, if any, in the stock solution of a core component running through the microchannel 1 are solid at room temperature, and preferably kept in a molten state by heating the microchannel 1. The heating temperature is preferably 50 to 100° C. At lower than 50° C., the reaction solvent may be solidified and unable to be delivered, whereas at higher than 100° C., the semiconductor crystals grow to disadvantageously broaden the particle size distribution of the resulting semiconductor crystals.

The third microchannel 3 is arranged to pass through an oil bath 4b disposed on a stirrer 5b for temperature control of a predetermined section of the microchannel 3. In the oil bath 4b, an immersion heater 11 for shells and a thermometer 6 connected to the temperature controller 9 are disposed.

Next, a method for producing the semiconductor nanocrystals with a core-shell structure using the system of FIG. 1 is explained, which is illustrative only and is not intended to limit the present invention.

First, the semiconductor material for the core component and the semiconductor material for the shell component are separately dissolved in a reaction solvent uniformly to prepare stock solutions of the core component and of the shell component, respectively. Then the stock solution of the core component is passed through the first microchannel 1 at a constant flow rate of 0.25 to 25 ml/min using the pump 10a. On the other hand, the stock solution of the shell component is simultaneously passed through the second microchannel 2 at a constant flow rate of 0.25 to 25 ml/min using the pump 10b.

Here, the predetermined section of the first microchannel 1 is maintained at 250 to 350° C. for forming the cores. Under these conditions, the cores of the semiconductor nanocrystals usually having a particle size of 1 to 6 nm are formed.

Subsequently, the streams of the stock solutions from the microchannels 1 and 2 merge to form a merged stream in the third microchannel 3. This merged stream is passed through the microchannel 3 at a constant flow rate of 0.5 to 50 ml/min, and maintained at 100 to 250° C. in the predetermined section mentioned above, so that the shell component grows epitaxially on the produced cores. The liquid discharged from the microchannel 3 is collected in a container and cooled, to eventually obtain the semiconductor nanocrystals having a particle size of preferably 1 to 10 nm and a full width at half maximum of not wider than 30 nm.

In sum, according to the method ofthe present invention, the semiconductor nanocrystals with a core-shell structure maybe produced in the system shown in FIG. 1 in the following way. First, the stock solution of the core component for forming the cores of the semiconductor nanocrystals is passed through the first microchannel 1, while the temperature for forming the cores is maintained at 250 to 350° C., thereby forming the cores in the liquid being delivered through the microchannel 1. Next, the shell component is epitaxially grown on the cores of the semiconductor nanocrystals by merging, in the third microchannel 3, the stream of the stock solution of the shell component from the second microchannel 2 with the stream from the microchannel 1, while the temperature of the merged stream is maintained at 100 to 250° C., thereby forming eventually the semiconductor nanocrystals having a desired core-shell structure.

The method of the present invention may be performed using a simple system as shown in FIG. 1.

According to the method of the present invention, semiconductor nanocrystals with a core-shell structure are obtained which usually have a particle size of 1 to 10 nm and a full width at half maximum of the fluorescence spectrum of not wider than 30 nm. The particle size may be measured with a transmission electron microscope, and the full width at half maximum of the fluorescence spectrum may be calculated from the spectrum measured by wavelength scan with a spectrofluorometer.

The semiconductor nanocrystals obtained by the present method, which are of high quality, are useful in applications in such fields as display elements, recording materials, optics, electronics, biological diagnosis, and the like. Further, the semiconductor nanocrystals obtained from step (3) may be coated on their surface with a polymer compound such as polyethylene glycol.

According to the method of the present invention, the semiconductor nanocrystals with a core-shell structure which have a particle size of 1 to 10 nm and a full width at half maximum of the fluorescence spectrum of not wider than 30 nm, may be produced continuously. By adjusting the production conditions, semiconductor nanocrystals with a core-shell structure having desired particle size and fluorescence wavelength suitable for their intended use, may be mass produced. Further, by arranging the microchannels used in the present method in a spiral shape, the production system may be made compact.

### EXAMPLES

The present invention will now be explained in more detail with reference to Examples, which are illustrative only and are not intended to limit the present invention.

### Example 1

(Preparation of Selenium Stock Solution)

525.8 mg of selenium (manufactured by WAKO PURE CHEMICALS INDUSTRIES, LTD., 99.999% purity) was measured out into a vial, which was then flushed with argon gas. 14 ml of dioctyl amine (manufactured by KISHIDA CHEMICAL CO., LTD.) and 2.83 ml of tributylphosphine (manufactured by ALDRICH CORPORATION) were added, and the mixture was irradiated with ultrasonic wave, to give a completely transparent solution.

(Preparation of Cadmium/Selenium Stock Solution)

203.7 mg of cadmium stearate (manufactured by WAKO PURE CHEMICALS INDUSTRIES, LTD.), 5.82 g of trioctylphosphine oxide (manufactured by ALDRICH CORPORATION, 99% purity), and 5.82 g of hexadecyl amine (manufactured by TOKYO KASEI KOGYOCO., LTD.) were measured out into a pear-shaped flask, which was then flushed with argon gas. The flask was placed in an oil bath at 70° C. to dissolve the contents, and 0.75 ml of a selenium stock solution previously prepared was added using syringes.

(Preparation of Zinc/Sulfur Stock Solution)

In a flask previously flushed with argon gas, 15 ml of tributylphosphine (manufactured by ALDRICH CORPORATION), 1.2 ml of 1M diethylzinc heptane solution (manufactured by ALDRICH CORPORATION), and 252 μl of bis(trimethylsilyl)sulfide (manufactured by FLUKA) were introduced.

(Production of CdSe—ZnS Semiconductor Nanocrystals)

CdSe—ZnS semiconductor nanocrystals were produced using the system shown in FIG. 1. Here, the lengths of the straight sections of the first, second, and third microchannels were 2 m, 0.1 m, and 2 m, respectively, and the inner diameters thereof were 600 μm, 1000 μm, and 1000 μm, respectively. The lengths of the heated sections of the first and third microchannels were both 1.8 m, and the lengths of the non-heated sections thereof were both 0.2 m. The temperature was set at room temperature. The microchannels were made of stainless steel.

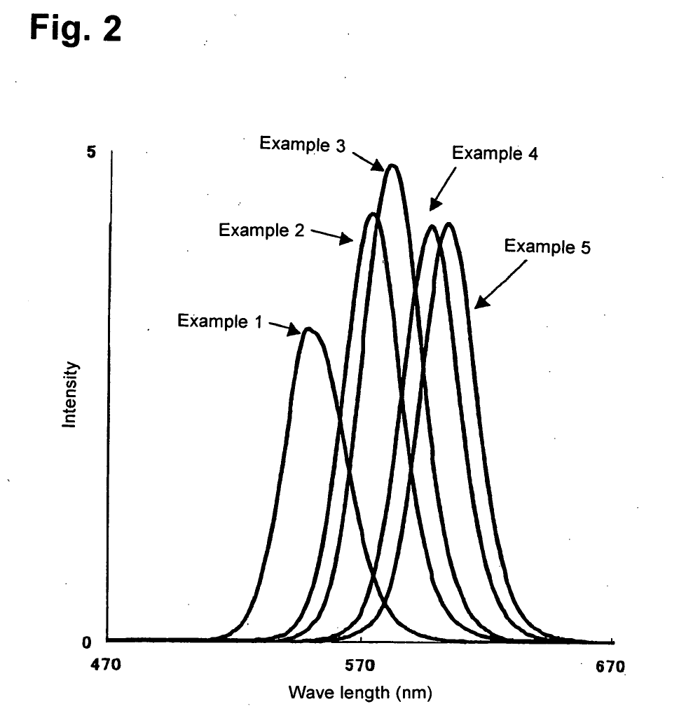

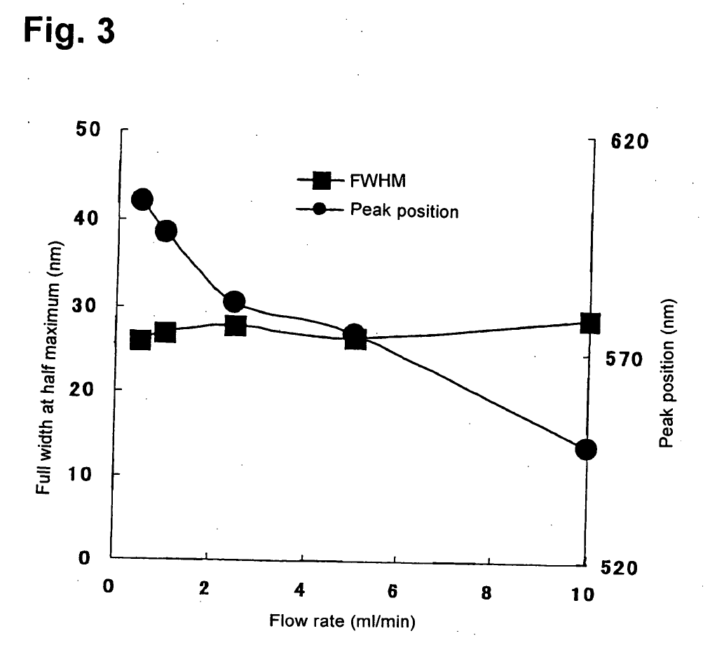

First, using a 50 ml syringe previously heated in a thermostatic chamber at 60° C., the entire amount of the cadmium/selenium stock solution was taken up, and the syringe was installed on a syringe pump (microfeeder, model JP-V-W7, manufactured by FURUE SCIENCE CO., LTD.). Since the cadmium/selenium stock solution solidifies at room temperature, ribbon heaters were immediately attached to keep the stock solution in a molten state under heating. Next, using another 50 ml syringe, the entire amount of the zinc/sulfur stock solution was taken up, and the syringe was installed on a syringe pump. The temperatures of the oil baths in the CdSe preparation section and in the ZnS coating section were set at 300° C. and 150° C., respectively, and the cadmium/selenium stock solution and the zinc/sulfur stock solution were fed at 10 ml/min. Incidentally, the first about 3 ml from the start of the feeding was not collected and discarded. The fluorescence spectrum of the thus obtained CdSe—ZnS was measured with a spectrofluorometer (model FP6300, manufactured by JASCO CORPORATION). The full width at half maximum (FWHM) and the peak position of the spectrum are shown in FIGS. 2 and 3, respectively.

The results were that the peak appeared at 548 nm, and the full width at half maximum was not wider than 30 nm, indicating that the obtained nanocrystals had a sharp fluorescence spectrum. The particle size of the obtained semiconductor nanocrystals was measured with a transmission electron microscope H-7000 (manufactured by HITACHI LTD.), and found to be 3.8 nm.

### Example 2

CdSe—ZnS semiconductor nanocrystals were prepared and subjected to the measurements in the same way as in Example 1, except that the delivery rate of the cadmium/selenium stock solution and the zinc/sulfur stock solution was changed from 10 ml/min to 5 ml/min. The full width at half maximum (FWHM) and the peak position of the fluorescence spectrum of the obtained CdSe—ZnS semiconductor nanocrystals are shown in FIGS. 2 and 3, respectively.

The results were that the peak appeared at 574 nm, and the full width at half maximum was not wider than 30 nm, indicating that the obtained nanocrystals had a sharp fluorescence spectrum. The particle size of the obtained semiconductor nanocrystals was found to be 4.1 nm.

### Example 3

CdSe—ZnS semiconductor nanocrystals were prepared and subjected to the measurements in the same way as in Example 1, except that the delivery rate of the cadmium/selenium stock solution and the zinc/sulfur stock solution was changed from 10 ml/min to 2.5 ml/min. The full width at half maximum (FWHM) and the peak position of the fluorescence spectrum of the obtained CdSe—ZnS semiconductor nanocrystals are shown in FIGS. 2 and 3, respectively.

The results were that the peak appeared at 581 nm, and the full width at half maximum was not wider than 30 nm, indicating that the obtained nanocrystals had a sharp fluorescence spectrum. The particle size of the obtained semiconductor nanocrystals was found to be 4.4 nm.

### Example 4

CdSe—ZnS semiconductor nanocrystals were prepared and subjected to the measurements in the same way as in Example 1, except that the delivery rate of the cadmium/selenium stock solution and the zinc/sulfur stock solution was changed from 10 ml/min to 1 ml/min. The full width at half maximum (FWHM) and the peak position of the fluorescence spectrum of the obtained CdSe—ZnS semiconductor nanocrystals are shown in FIGS. 2 and 3, respectively.

The results were that the peak appeared at 597 nm, and the full width at half maximum was not wider than 30 nm, indicating that the obtained nanocrystals had a sharp fluorescence spectrum. The particle size of the obtained semiconductor nanocrystals was found to be 4.8 nm.

### Example 5

CdSe—ZnS semiconductor nanocrystals were prepared and subjected to the measurements in the same way as in Example 1, except that the delivery rate of the cadmium/selenium stock solution and the zinc/sulfur stock solution was changed from 10 ml/min to 0.5 ml/min. The full width at half maximum (FWHM) and the peak position of the fluorescence spectrum of the obtained CdSe—ZnS semiconductor nanocrystals are shown in FIGS. 2 and 3, respectively.

The results were that the peak appeared at 604 nm, and the full width at half maximum was not wider than 30 nm, indicating that the obtained nanocrystals had a sharp fluorescence spectrum. The particle size of the obtained semiconductor nanocrystals was found to be 5.2 nm.

In the above Examples, it was demonstrated that, by the method of the present invention, semiconductor nanocrystals with a core-shell structure having a particle size of 1 to 10 nm were mass produced continuously and easily. From FIG. 2, it is understood that the method of the present invention provides semiconductor nanocrystals having a full width at half maximum of the fluorescence spectrum of not wider than 30 nm and composed of monodisperse particle with a sharp fluorescence spectrum. From FIG. 3, it is understood that, by adjusting the flow rate in the present method, semiconductor nanocrystals having different full widths at half maximum and different peaks may be produced.

### Example 6

Preparation of Polyethylene Glycol-Modified CdSe—ZnS Semiconductor Nanocrystals

In a 50 ml pear-shaped flask, 500 mg of polyethylene glycol having a thiol group at one end and methoxy at the other end and having a number average molecular weight of 5000, and 16.5 mg of cadmium chloride were introduced, and 10 ml of a phosphate buffer was added to dissolve these components. Then a magnetic stirrer and 5 ml of chloroform were introduced into the flask, and the flask was attached to the discharge port of the reaction mixture in the system shown in FIG. 1.

1 ml of the reaction liquid was collected in the pear-shaped flask, stirred for 1 hour at room temperature, mixed with 20 ml of hexane, and left to stand. Upon irradiation with a 254 nm UV lamp, fluorescence was observed only in the lower phase, which was the phosphate buffer phase.

From the above result, the obtained crystals were found to be polyethylene glycol-modified CdSe—ZnS semiconductor nanocrystals, and dispersible in an aqueous phase.

1. A method for producing semiconductor nanocrystals with a core-shell structure comprising the steps of:
(1) passing a stock solution of a core component consisting of CdX, wherein X stands for S, Se, or Te, through a first hollow microchannel having an inner diameter of 1 to 1000 μm at a constant flow rate of 0.25 to 25 ml/min to form cores of semiconductor nanocrystals in a temperature range of 250 to 350° C.;
(2) passing a stock solution of a shell component consisting of ZnR, wherein R stands for S, Se, Te, or O, through a second hollow microchannel having an inner diameter of 1 to 1000 μm;
(3) passing a stream of said cores formed through said first microchannel merged with a stream of said shell component from said second microchannel, through a third hollow microchannel having an inner diameter of 1 to 1000 μm at a constant flow rate of 0.5 to 50 ml/min to epitaxially grow said shell component on said cores in a temperature range of 100 to 250° C., to thereby form a core-shell structure,
wherein said first, second, and third microchannels communicate with each other, and
wherein said step (3) is performed consecutively to said steps (1) and (2).

2. The method of claim 1, wherein said first microchannel in step (1) and said third microchannel in step (3) are 0.1 to 10 m long, and arranged in a spiral shape.

3. Semiconductor nanocrystals obtained by the method of claim 1, said nanocrystals having a core consisting of CdX, wherein X stands for S, Se, or Te, and a shell consisting of ZnR, wherein R stands for S, Se, Te, or O, said nanocrystals having a particle size of 1 to 10 nm, and a full width at half maximum of the fluorescence spectrum of not wider than 30 nm.
